SEQUENCE LISTING (14 sequences)

SEQ ID NO: 1 - protein, 718 aa, Artificial sequence
  Synthetic
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 661 aa, Artificial sequence
  Synthetic
MNNLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKLPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 461 aa, Homo sapiens [sequence omitted]

SEQ ID NO: 5 - protein, 401 aa, Homo sapiens
MNKLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 6 - DNA, 40 bp, Artificial sequence
  Synthetic
aggtatagcggccgccaccatgaacaacttgctgtgctgc

SEQ ID NO: 7 - DNA, 46 bp, Artificial sequence
  Synthetic
gtaaatgccacctgggcgggcaatttttgagttgattcactgtttc

SEQ ID NO: 8 - DNA, 23 bp, Artificial sequence
  Synthetic
ttgcccgcccaggtggcatttac

SEQ ID NO: 9 - DNA, 43 bp, Artificial sequence
  Synthetic
tggtggtgtctagagactcatttacccggagacagggagaggc

SEQ ID NO: 10 - DNA, 29 bp, Artificial sequence
  Synthetic
aagcttgcggccgcgagctcggatccact

SEQ ID NO: 11 - DNA, 20 bp, Artificial sequence
  Synthetic
gtcgccagtgctcccttcag

SEQ ID NO: 12 - DNA, 42 bp, Artificial sequence
  Synthetic
ctgaagggagcactggcgacgaaacgtttcctccaaagtacc

SEQ ID NO: 13 - DNA, 23 bp, Artificial sequence
  Synthetic
tttttgagttgattcactgtttc

SEQ ID NO: 14 - DNA, 41 bp, Artificial sequence
  Synthetic
gaaacagtgaatcaactcaaaaatccacgtcccccacccgg

(19) United States
(12) Patent Application Publication
(10) Pub. No.: US 2023/0203204 A1
(43) Pub. Date: Jun. 29, 2023

(54) FUSION PROTEIN, PREPARATION METHOD THEREFOR AND USE THEREOF

(71) Applicant: Bang Ding, Shanghai (CN)

(72) Inventors: Zeling Cai, Shanghai (CN); Yi Chen, Shanghai (CN)

(73) Assignee: Bang Ding, Shanghai (CN)

(21) Appl. No.: 17/792,700

(22) PCT Filed: Jan. 13, 2021
(86) PCT No.: PCT/CN2021/071544

§ 371 Date: Feb. 8, 2023

(30) Foreign Application Priority Data
Jan. 13, 2020 (WO) PCT/CN2020/071772

(51) Int. Cl. C07K 19/00; A61K 38/16; C07K 14/715; C12N 15/09; A61P 19/10; A61P 37/00

(52) U.S. Cl. CPC C07K 19/00; A61K 38/16; C07K 14/715; C12N 15/09; A61P 19/10; A61P 37/00

(57) ABSTRACT

Provided are a fusion protein, a preparation method therefor and use thereof. The protein can: (a) inhibit TNFa-induced apoptosis; and/or (b) inhibit TRAIL-induced apoptosis; and/or (c) inhibit differentiation of RANKL-induced mononuclear macrophages. The protein is capable of (i) preventing and/or treating infectious diseases; and/or (ii) preventing and/or treating autoimmune diseases; and/or (iii) preventing and/or treating osteoporosis or loss; and/or (iv) preventing and/or treating tumor-associated diseases.

### TECHNICAL FIELD

The present invention relates to the field of biology and medicine, and more particularly to a fusion protein and its preparation method and use.

### BACKGROUND

Autoimmune disease is an immune-mediated disease in which the immune system responds positively to the host's self-antigens, causing pathological damage to tissues or organs, affecting their physiological functions, and ultimately leading to various clinical symptoms. Rheumatoid arthritis (RA) is one of the most common autoimmune diseases with the highest incidence, causing damage to the synovium, articular cartilage and bones. RA is caused and maintained by the interaction of a series of pro-inflammatory cytokines, among which TNFα is a key inflammation-mediated cytokine in patients with inflammatory arthritis. At present, there are a variety of drugs that inhibit the function of TNFa (TNFa blocking agent) in clinical treatment of various autoimmune diseases.

Arthritis is chronic soft tissue inflammation that usually accompanies by the destruction of the joint at last. Immune-mediated arthritis often results in focal bone erosions in inflamed joints, mainly due to excessive osteoclast activity mediated by the receptor activator NF-κB ligand (RANKL). RANKL is an important mediator of bone resorption, osteoprotegrin (OPG) is a decoy receptor of RANKL, a soluble protein that can specifically bind to RANKL, and is a physiological inhibitor of RANKL.

However, although current drugs can improve inflammation in RA patients. However, there is no or very limited preventive and therapeutic effect on focal bone erosion and systemic bone loss in inflamed joints.

Therefore, there is an urgent need in the art to develop drugs that can not only improve inflammation in RA patients, but also prevent focal bone erosion and systemic bone loss caused by inflammation.

### SUMMARY OF THE INVENTION

The purpose of the present invention is to provide a drug that can not only improve inflammation in RA patients, but also prevent focal bone erosion and systemic bone loss caused by inflammation.

In a first aspect of the present invention, it provides a fusion protein comprising the following elements fused together: (a) a TNF receptor or an active fragment thereof; (b) OPG or an active fragment thereof; and optionally (c) Fc fragment.

In another preferred embodiment, the fusion protein retains the biological activities of the above elements (a) and (b).

In another preferred embodiment, the TNF receptor is selected from the group consisting of TNFR2, TNFR1, and a combination thereof.

In another preferred embodiment, the TNF receptor is derived from a human or non-human mammal, more preferably from a rodent (e.g., a mouse, rat), primate and human.

In another preferred embodiment, the TNF receptor includes wild type and mutant type.

In another preferred embodiment, the TNF receptor includes a full-length, mature form of TNF receptor, or an active fragment thereof.

In another preferred embodiment, the TNF receptor also includes a derivative of TNF receptor.

In another preferred embodiment, the derivative of TNF receptor includes a modified TNF receptor, protein molecule with amino acid sequence homologous to natural TNF receptor and with natural TNF receptor activity, dimers or multimers of TNF receptor, fusion protein containing the amino acid sequence of TNF receptor.

In another preferred embodiment, the modified TNF receptor is a PEGylated TNF receptor.

In another preferred embodiment, the ″ protein molecule with amino acid sequence homologous to natural TNF receptor and with natural TNF receptor activity ″ means that a protein molecule whose amino acid sequence has ≥85% homology, preferably ≥90% homology, more preferably ≥95% homology, most preferably ≥98% homology with TNF receptor and has the activity of TNF receptor.

In another preferred embodiment, the TNF receptor includes a first domain, a second domain, a third domain, and/or a fourth domain.

In another preferred embodiment, the first domain, the second domain, the third domain, and/or the fourth domain are each independently a cysteine-rich region (CRD).

In another preferred embodiment, the TNF receptor is the second domain and the third domain of the TNF receptor extracellular domain, or the TNF receptor extracellular domain containing the second domain and the third domain.

In another preferred embodiment, the TNF receptor contains or has positions 1-257, or positions 23-257, or positions 77-162, or positions 205-257 of the amino acid sequence of TNFR2 (SEQ ID NO.: 4).

In another preferred embodiment, the OPG is derived from a human or non-human mammal, more preferably from a rodent (e.g., mouse, rat), primate and human.

In another preferred embodiment, the OPG includes wild type and mutant type.

In another preferred embodiment, the OPG includes full-length, mature form of OPG, or an active fragment thereof.

In another preferred embodiment, the OPG also includes a derivative of OPG.

In another preferred embodiment, the derivative of OPG include modified OPG, a protein molecule whose amino acid sequence is homologous to natural OPG and have natural OPG activity, dimers or multimers of OPG, and a fusion protein containing the OPG amino acid sequence.

In another preferred embodiment, the modified OPG is PEGylated OPG. In another preferred example, the “protein molecule whose amino acid sequence is homologous to natural OPG and have natural OPG activity,” means that a protein molecule whose amino acid sequence has 85% homology, preferably 90% homology, more preferably ≥95% homology, most preferably ≥98% homology with OPG and has the activity of OPG.

In another preferred embodiment, the OPG includes a first domain, a second domain, a third domain, and/or a fourth domain.

In another preferred embodiment, the first domain, the second domain, the third domain, and/or the fourth domain are each independently a cysteine-rich region (CRD).

In another preferred embodiment, the OPG is the second, third and fourth domain of the OPG extracellular region, or the OPG extracellular region containing the second domain, the third domain, and the fourth domain.

In another preferred embodiment, the OPG contains or has positions 1-194, or positions 22-194, or positions 22-194 of the OPG amino acid sequence (SEQ ID NO.: 5).

In another preferred embodiment, the Fc fragment is derived from a human or non-human mammal, more preferably from a rodent (e.g., mouse, rat), primate and human.

In another preferred embodiment, the Fc fragment is the Fc fragment of immunoglobulin IgG, preferably the Fc part of IgG1.

In another preferred embodiment, the Fc fragment includes a natural Fc fragment and Fc mutant.

In another preferred embodiment, the Fc fragment contains or has positions 99(Glu)-330(Lys) of the amino acid sequence of human IgG1 (accession number: UniProtKB -P01857).

In another preferred embodiment, the Fc fragment contains or has positions 487-718 of SEQ ID NO.:1.

In another preferred embodiment, the Fc fragment contains or has positions 430-661 of SEQ ID NO.:2.

In another preferred embodiment, the amino acid sequence of the Fc fragment is shown in SEQ ID NO.:3.

In another preferred embodiment, the fusion protein has the structure as shown in the following Formula I or II:

X-Y-Z (I)

Y-X-Z (II);

wherein

X is a TNF receptor or an active fragment thereof;

Y is OPG or an active fragment thereof;

Z is an optional Fc fragment; “-” represents a peptide bond or peptide linker connecting the above elements.

In another preferred embodiment, any two of the X, Y, and Z are connected in a head-to-head, head-to-tail, tail-to-head or tail-to-tail manner.

In another preferred embodiment, the “head” refers to the N-terminus of a polypeptide or a fragment thereof, especially the N-terminus of a wild-type polypeptide or a fragment thereof.

In another preferred embodiment, the “tail” refers to the C-terminus of a polypeptide or a fragment thereof, especially the C-terminus of a wild-type polypeptide or a fragment thereof.

In another preferred embodiment, the length of the peptide linker is 0-20 amino acids, preferably 0-10 amino acids.

In another preferred embodiment, the fusion protein is a homodimer.

In another preferred embodiment, the fusion protein is selected from the group consisting of:

(A) a polypeptide having the amino acid sequence as shown in SEQ ID NO: 1 or 2;

(B) a polypeptide having ≥80% homology (preferably, ≥90% homology; preferably ≥95% homology; most preferably, ≥97% homology, such as 98% or more, 99% or more) with the amino acid sequence as shown in SEQ ID NO: 1 or 2, and the polypeptide has (a) inflammation inhibitory activity or (b) osteoporosis and/or bone loss inhibitory activity;

(C) a derivative polypeptide formed by the amino acid sequence as shown in SEQ ID NO.: 1 or 2 through the substitution, deletion or addition of 1-5 amino acid residues and retains (a) inflammation inhibitory activity or (b) osteoporosis and/or bone loss inhibitory activity.

In another preferred embodiment, the amino acid sequence of the fusion protein is shown in SEQ ID NO.: 1 or 2.

In a second aspect of the present invention, it provides an isolated polynucleotide encoding the fusion protein of the first aspect of the present invention.

In another preferred embodiment, the polynucleotide additionally contains an auxiliary element selected from the group consisting of a signal peptide, a secretory peptide, a tag sequence (e.g., 6His), and a combination thereof, flanking the ORF of the mutein or fusion protein.

In another preferred embodiment, the polynucleotide is selected from the group consisting of DNA sequence, RNA sequence, and a combination thereof.

In a third aspect of the present invention, it provides a vector, which contains the polynucleotide as described in the second aspect of the present invention.

In another preferred embodiment, the vector comprises one or more promoters operably linked to the nucleic acid sequence, enhancer, transcription termination signal, polyadenylation sequence, origin of replication, selectable marker, nucleic acid restriction site, and/or homologous recombination site.

In another preferred embodiment, the vector includes a plasmid and viral vector.

In another preferred embodiment, the viral vector is selected from the group consisting of adeno-associated virus (AAV), adenovirus, lentivirus, retrovirus, herpes virus, SV40, poxvirus, and a combination thereof.

In another preferred embodiment, the vector includes an expression vector, a shuttle vector, and an integration vector.

In a fourth aspect of the present invention, it provides a host cell, wherein the host cell contains the vector of the third aspect of the present invention, or the polynucleotide of the second aspect of the present invention is integrated into its genome.

In another preferred embodiment, the host cell is a eukaryotic cell, such as a yeast cell, plant cell or mammalian cell (including a human and non-human mammal).

In another preferred embodiment, the host cell is a prokaryotic cell, such as Escherichia coli.

In another preferred embodiment, the yeast cell is selected from the group of yeasts from one or more sources: Pichia pastoris, Kluyveromyces and a combination thereof; preferably, the yeast cell includes: Kluyveromyces, more preferably Kluyveromyces marxianus, and/or Kluyveromyces lactis.

In another preferred embodiment, the host cell is selected from the group consisting of Escherichia coli, wheat germ cells, insect cells, SF9, SP2/0, Hela, HEK293, CHO (such as CHOKS), yeast cells, and a combination thereof.

In a fifth aspect of the present invention, it provides a method for producing the fusion protein of the first aspect of the present invention, the method comprising the steps of:

Under conditions suitable for expression, culturing the host cell according to the fourth aspect of the present invention, thereby expressing the fusion protein; and/or

Isolating or purifying the fusion protein.

In a sixth aspect of the present invention, it provides a pharmaceutical composition comprising the fusion protein of the first aspect of the present invention and a pharmaceutically acceptable carrier thereof.

In another preferred embodiment, the pharmaceutical composition further includes other drugs for inhibiting inflammatory activity.

In another preferred embodiment, other drugs for inhibiting inflammatory activity are selected from the group consisting of hormone drugs, non-steroid anti-inflammatory drugs, immunosuppressive drugs, small molecule targeted drugs, biological agents, and a combination thereof.

In another preferred embodiment, the hormonal drugs are selected from the group consisting of hydrocortisone, prednisone, prednisolone, dexamethasone, and a combination thereof.

In another preferred embodiment, the non-steroid anti-inflammatory drugs are selected from the group consisting of aspirin, indometacin, naproxen, ibuprofen, diclofenac, loxoprofen, meloxicam, celecoxib, etoricoxib, parecoxib, and a combination thereof.

In another preferred embodiment, the immunosuppressive drugs are selected from the group consisting of methotrexate, cyclophosphamide, azathioprine, cyclosporine, mycophenolate mofetil, tacrolimus, sirolimus, leflunomide and a combination thereof.

In another preferred embodiment, the small molecule targeted drugs are selected from the group consisting of tofacitinib, baricitinib, and a combination thereof.

In another preferred embodiment, the biological agents are selected from the group consisting of etanercept, certolizumab, adalimumab, golimumab, infliximab, tocilizumab, Secukinumab, Eutecumab, canakinumab, anakinra, Rilonacept, abatacept, rituximab, belimumab, and a combination thereof.

In a seventh aspect of the present invention, it provides a use of the fusion protein as described in the first aspect of the present invention, the polynucleotide as described in the second aspect of the present invention, the vector as described in the third aspect of the present invention, and the host cell as described in the fourth aspect of the present invention for the preparation of a composition or preparation for (i) preventing and/or treating infectious diseases; and/or (ii) preventing and/or treating autoimmune diseases; and/or (iii) preventing and/or treating osteoporosis or loss; and/or (iv) preventing and/or treating tumor-related diseases.

In another preferred embodiment, the infectious disease includes septic shock (such as LPS-induced septic shock).

In another preferred embodiment, the autoimmune disease is selected from the group consisting of rheumatoid arthritis, ankylosing spondylitis, and a combination thereof.

In another preferred embodiment, the osteoporosis or loss is selected from the group consisting of osteoporosis or/and loss caused by rheumatoid arthritis, osteoporosis in postmenopausal women, and a combination thereof.

In another preferred embodiment, the tumor-related disease is selected from the group consisting of multiple myeloma, bone-related bone metastases solid tumor, and a combination thereof.

In another preferred embodiment, the composition or preparation is also used for one or more purposes selected from the group consisiting of:

(a) the inhibition of TNFa-induced apoptosis;

(b) the inhibition of TNFa-induced immune activation;

(c) the inhibition of TRAIL-induced apoptosis;

(d) the inhibition of RANKL-induced monocyte-macrophage differentiation;

(e) the inhibition of RANKL-induced osteoclastogenesis.

In another preferred embodiment, the cell is selected from the group consisting of fibroblast, mononuclear macrophage, vascular endothelial cell, and a combination thereof.

In another preferred embodiment, the composition is a pharmaceutical composition.

In an eighth aspect of the present invention, it provides a method for inhibiting cell apoptosis, comprising the steps of:

In the presence of the fusion protein as described in the first aspect of the present invention, culturing a cell, thereby inhibiting cell apoptosis.

In another preferred embodiment, the cell is selected from the group consisting of fibroblast, haematological tumor cell, solid tumor cell, and a combination thereof.

In another preferred embodiment, the haematological tumor cell is selected from the group consisting of B-cell malignant cell, acute myeloid leukemia cell, Hodgkin's lymphoma cell, T-cell malignant cell, multiple myeloma cell, and a combination thereof.

In another preferred embodiment, the solid tumor cell is selected from the group consisting of breast cancer cell, non-small cell lung cancer cell, hepatoma carcinoma cell, colon cancer cell, gastric cancer cell, and a combination thereof.

In another preferred embodiment, the cell is a cell cultured in vitro.

In another preferred embodiment, the method is non-therapeutic and non-diagnostic.

In another preferred embodiment, the apoptosis includes TNFa-induced apoptosis; and/or TRAIL-induced apoptosis.

In a ninth aspect of the present invention, it provides a method for inhibiting the differentiation of mononuclear macrophages, comprising the steps of:

In the presence of the fusion protein as described in the first aspect of the present invention, culturing a mononuclear macrophage, thereby inhibiting the differentiation of the mononuclear macrophage.

In another preferred embodiment, the mononuclear macrophage is a cell cultured in vitro.

In another preferred embodiment, the method is non-therapeutic and non-diagnostic.

In another preferred embodiment, the mononuclear macrophage differentiation is RANKL-induced mononuclear macrophage differentiation.

In a tenth aspect of the present invention, it provides a method for preventing and/or treating diseases, comprising the steps of: administering the fusion protein as described in the first aspect of the present invention to a subject in need.

In another preferred embodiment, the fusion protein is administered in the form of monomers and/or dimers.

In another preferred embodiment, the subject is a human.

In another preferred embodiment, the disease is selected from the group consisting of infectious disease, autoimmune disease, osteoporosis or loss, tumor-related disease, and a combination thereof.

It should be understood that, within the scope of the present invention, the technical features specifically described above and below (such as in the Examples) can be combined with each other, thereby constituting a new or preferred technical solution which needs not be described one by one.

### DESCRIPTION OF THE DRAWINGS

FIG. 1 is a schematic diagram of the structures of fusion proteins OPG-TNFR2-Fc and TNFR2-OPG-Fc. OPG-TNFR2-Fc contains (from N-terminus to C-terminus): OPG amino acid sequence 22-194 including 4 CRDs, TNFR2 amino acid sequence 23-257 including 4 CRDs, and IgG1 Fc. TNFR2-OPG-Fc contains (from N-terminus to C-terminus): TNFR2 amino acid sequence 23-257 including 4 CRDs, OPG amino acid sequence 22-194 including 4 CRDs, TNFR2 amino acid sequence 203-257, and IgG1 Fc.

FIG. 2 shows reduced and non-reduced denatured protein gel SDS-PAGE analysis of fusion proteins. Fusion proteins purified by Protein A affinity chromatography are analyzed and identified by 6% non-reduced denatured SDS-PAGE (A) and 10% reduced denatured SDS-PAGE (B). 1: Human IgG1; 2: OPG-TNFR2-Fc; 3: TNFR2-OPG-Fc. Load 5 mg per sample.

FIG. 3 shows HPLC-SEC analysis of fusion proteins. The fusion protein is analyzed by HPLC-SEC (TSKgel G3000SWXL) chromatographic column. The red curve represents OPG-TNFR2-Fc; the green curve represents TNFR2-OPG-Fc. Molecular weights are indicated by the blue curve.

FIG. 4 shows the results of an ELISA study of in vitro binding to recombinant human TNFa.

FIG. 5 shows the results of an ELISA study of in vitro binding to recombinant human RANKL.

FIG. 6 shows the results of an ELISA study of binding recombinant human TRAIL in vitro.

FIG. 7 shows the results of a study on inhibition of TNFa-induced apoptosis in L929 cells.

FIG. 8 shows the results of a study on inhibition of Trail-induced apoptosis in L929 cells.

FIG. 9 shows the results of a cell differentiation study of inhibiting RANKL-induced RAW264.7 cells.

FIG. 10 shows the results of a study to inhibit LPS-induced septic shock death in mice.

FIG. 11 shows (A): reduction in the incidence of CIA-induced inflammation in mice; (B) reduction in the degree of inflammation in CIA-induced mice.

### DETAILED DESCRIPTION

After intensive research, the inventors have unexpectedly found that by fusing (a) TNF receptor or an active fragment thereof; (b) OPG or an active fragment thereof; and optional (c) Fc fragment, the obtained fusion protein has extremely excellent biological activity, and can very significantly (a) inhibit TNFa-induced apoptosis; and/or (b) inhibit TRAIL-induced apoptosis; and/or (c) inhibit RANKL-induced differentiation of mononuclear macrophage. In addition, the fusion protein has good stability and long half-life, thus helping to (i) prevent and/or treat infectious diseases; and/or (ii) prevent and/or treat autoimmune diseases; and/or (iii) prevent and/or treat tumor-related diseases. The present invention has been completed on this basis.

As used herein, unless otherwise specified, Fc refers to the Fc fragment of a human immunoglobulin. The term “immunoglobulin Fc region” refers to the constant region of an immunoglobulin chain, particularly the carboxy-terminus or a part thereof, of the constant region of an immunoglobulin heavy chain, for example, an immunoglobulin Fc region may comprise a combination of two or more domains of heavy chain CH1, CH2, CH3 and an immunoglobulin hinge region, in a preferred embodiment, the Fc region of the immunoglobulin used includes at least one immunoglobulin hinge region, one CH2 domain and one CH3 domain, preferably lacking CH1 domain.

It is known that there are various classes of human immunoglobulins, such as IgA, IgD, IgE, IgM and IgG (including four subclasses of IgG1, IgG2, IgG3, and IgG4). The selection of a particular immunoglobulin Fc region from a particular immunoglobulin class and subclass is within the purview of those skilled in the art an in a preferred example, the Fc region of an immunoglobulin can select a coding sequence comprising the Fc region of a human immunoglobulin IgG4 subclass, which deletes an immunoglobulin heavy chain 1 domain (CH1), but includes the hinge region and the coding sequences of CH2, CH3, and two domains.

As used herein, the words “comprising”, “having” or “including” include “comprising”, “consisting mainly of”, “consisting essentially of”, and “consisting of”; “consisting mainly of”, “consisting essentially of” and “consisting of” are subordinate concepts of “comprises”, “has” or “includes”.

As used herein, unless otherwise stated, the fusion protein is an isolated protein, not associated with other proteins, polypeptides or molecules, either as a purified product of recombinant host cell culture or as a purified extract.

TNF Receptor or an Active Fragment Thereof

TNFR1 and 2 belong to the TNF receptor superfamily members and are type I transmembrane proteins. The molecular weights of TNFR1 and TNFR2 are 55 and 75 kDa, respectively, and their extracellular regions each contain 4 cysteine-rich regions, and the amino acid sequences are 23% identical. TNFR1 is widely expressed in various types of cells, while TNFR2 is mainly expressed in hematopoietic cells, such as T cells and natural killer cells, as well as endothelial cells, nerve cells, thymocytes and mesenchymal stem cells. TNFR1 mainly binds soluble TNFa, while TNFR2 binds soluble TNFa and can also bind TNFa in membrane protein form.

In the present invention, TNF receptors include, but are not limited to, TNFR2, TNFR1. As used herein, unless otherwise specified, TNF-R2, TNFR II, hTNFR II are used interchangeably and refer to the human tumor necrosis factor type II receptor.

OPG

OPG (osteoprotegrin) is a secreted protein with a molecular weight of about 55-60 kDa, which has the functions of inhibiting osteoclastogenesis and regulating bone density. OPG is a member of the tumor necrosis factor receptor superfamily (TNFR), and mature OPG contains 4 cysteine-rich regions, 2 death domains and 1 heparin binding domain. The cysteine-rich region is critical for the interaction of OPG with its ligands, and the C-terminal cystein mediates the protein to form homodimers. OPG is widely and persistently expressed in mesenchymal stem cells, fibroblasts and endothelial cells. OPG, also known as the decoy receptor for TNF superfamily ligands, binds to RANKL and TRAIL. TRAIL reduces OPG release from expressing cells, whereas OPG inhibits TRAIL-induced apoptosis. OPG can inhibit the function of RANKL to promote osteoclastogenesis and promote osteoclastogenesis. Lack of OPG in the human body causes juvenile Paget disease, and when there is insufficient OPG to balance RANKL and RANK functions, it can lead to osteoporosis and vascular calcification.

Fusion Protein

As used herein, “fusion protein of the present invention”, or “polypeptide” refers to the fusion protein of the first aspect of the present invention.

In a preferred embodiment, the fusion protein of the present invention comprises the following elements: (a) TNF receptor or an active fragment thereof, (b) OPG or an active fragment thereof, and optionally (c) an Fc fragment. In the fusion protein of the present invention, the elements (e.g., between element a and element b, element b or element c) may contain or not contain linker sequences. The linker sequence is usually a sequence that has no effect on the two proteins.

In another preferred example, the structure of the fusion protein is shown as X-Y-Z (I) or Y-X-Z (II), wherein X is TNF receptor or its active fragment; Y is OPG or its active fragment; Z is optional Fc fragment.

In another preferred embodiment, the fusion protein has the amino acid sequence shown in SEQ ID NO.: 1 or 2.

As used herein, the term “fusion protein” also includes variant forms of fusion proteins having the above-mentioned activities (sequences as shown in SEQ ID NO.: 1 or 2). These variants include (but are not limited to): deletions, insertions and/or substitutions of 1-3 (usually 1-2, more preferably 1) amino acids, as well as addition or deletion of one or several (usually within 3, preferably within 2, more preferably within 1) amino acids at the C-terminal and/or N-terminal. For example, in the art, substitution with amino acids of close or similar properties generally does not alter the function of the protein. For another example, addition or deletion of one or several amino acids at the C-terminus and/or N-terminus usually does not alter the structure and function of the protein. Furthermore, the term also includes monomeric and multimeric forms of the polypeptides of the invention. The term also includes linear as well as nonlinear polypeptides (e.g., cyclic peptides).

The present invention also includes active fragments, derivatives and analogs of the above fusion proteins. As used herein, the terms “fragment”, “derivative” and “analog” refer to polypeptides that substantially retain the function or activity of the fusion proteins of the present invention. The polypeptide fragments, derivatives or analogs of the present invention may be (i) a polypeptide in which one or several conservative or non-conservative amino acid residues (preferably conservative amino acid residues) have been substituted, or (ii) a polypeptide having a substituent group in one or more amino acid residues, or (iii) a polypeptide formed by the fusion of an antigenic peptide with another compound (such as a compound that prolongs the half-life of a polypeptide, such as polyethylene glycol) or (iv) a polypeptide formed by fusing an additional amino acid sequence with this polypeptide sequence (a fusion protein formed by fusing with a leader sequence, a secretory sequence, or a tag sequence such as 6xHis). These fragments, derivatives and analogs are well known to those skilled in the art in light of the teachings herein.

A class of preferred active derivatives refers to that compared with the amino acid sequence of Formula I or Formula II , at most 3, preferably at most 2, more preferably at most 1 amino acid are replaced by amino acids with close or similar properties to form a polypeptide. These conservatively variant polypeptides are best produced by amino acid substitutions according to Table A.

TABLE A

| | | Preferred |
|---|---|---|
| initial residue | representative substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The present invention also provides analogs of the fusion proteins of the present invention. The differences between these analogs and the polypeptides shown in SEQ ID NO.: 1 or SEQ ID NO.: 2 may be differences in amino acid sequence, differences in modified forms that do not affect the sequence, or both. Analogs also include analogs with residues other than natural L-amino acids (e.g., D-amino acids), as well as analogs with non-naturally occurring or synthetic amino acids (e.g., β, γ-amino acids). It should be understood that the polypeptides of the present invention are not limited to the representative polypeptides exemplified above.

Modified (generally without altering the primary structure) forms include chemically derivatized forms such as acetylation or carboxylation of the polypeptide in vivo or in vitro. Modifications also include glycosylation, such as those resulting from glycosylation modifications in the synthesis and processing of the polypeptide or in further processing steps. Such modifications can be accomplished by exposing the polypeptide to enzymes that perform glycosylation, such as mammalian glycosylases or deglycosylases. Modified forms also include sequences with phosphorylated amino acid residues (e.g., phosphotyrosine, phosphoserine, phosphothreonine). Also included are polypeptides that have been modified to increase their resistance to proteolysis or to optimize their solubility properties.

Expression Vectors and Host Cells

The present invention also relates to vectors comprising the polynucleotides of the present invention, as well as host cells produced by genetic engineering with the vectors of the present invention or the coding sequences of fusion proteins of the present invention, and methods for producing the polypeptides of the present invention by recombinant techniques.

The polynucleotide sequences of the present invention can be used to express or produce recombinant fusion proteins by conventional recombinant DNA techniques. Generally there are the following steps:

(1). Use the polynucleotide (or variant) encoding the fusion protein of the present invention of the present invention, or transform or transduce a suitable host cell with a recombinant expression vector containing the polynucleotide;

(2). Host cells cultured in a suitable medium;

(3). Separation and purification of proteins from culture medium or cells.

In the present invention, the polynucleotide sequence encoding the fusion protein can be inserted into a recombinant expression vector. The term “recombinant expression vector” refers to bacterial plasmids, bacteriophages, yeast plasmids, plant cell viruses, mammalian cell viruses such as adenoviruses, retroviruses, or other vectors well known in the art. Any plasmids and vectors can be used as long as they are replicable and stable in the host. An important feature of expression vectors is that they typically contain an origin of replication, a promoter, marker genes and translational control elements.

Methods well known to those skilled in the art can be used to construct expression vectors containing the DNA sequences encoding the fusion proteins of the invention and appropriate transcriptional/translational control signals. These methods include in vitro recombinant DNA technology, DNA synthesis technology, in vivo recombinant technology, and the like. The DNA sequence can be operably linked to an appropriate promoter in an expression vector to direct mRNA synthesis. Representative examples of these promoters are: the lac or trp promoter of E. coli; the λ phage PL promoter; eukaryotic promoters including the CMV immediate early promoter, the HSV thymidine kinase promoter, the early and late SV40 promoters, the Retroviral LTRs and some other known promoters that control the expression of genes in prokaryotic or eukaryotic cells or their viruses. Expression vectors also include a ribosome binding site for translation initiation and a transcription terminator.

In addition, the expression vector preferably contains one or more selectable marker genes to provide phenotypic traits for selection of transformed host cells, such as dihydrofolate reductase for eukaryotic cell culture, neomycin resistance, and green fluorescent protein (GFP), or Tetracycline or ampicillin resistance for E. coli.

Vectors comprising the appropriate DNA sequences described above, together with appropriate promoter or control sequences, can be used to transform appropriate host cells so that they can express the protein.

Host cells can be prokaryotic cells (e.g., E. coli), or lower eukaryotic cells, or higher eukaryotic cells, such as yeast cells, plant cells, or mammalian cells (including humans and non-human mammals). Representative examples are: Escherichia coli, wheat germ cells, insect cells, SF9, Hela, HEK293, CHO, yeast cells, etc. In a preferred embodiment of the present invention, a yeast cell (such as Pichia pastoris, Kluyveromyces, or a combination thereof is selected; preferably, the yeast cells include: Kluyveromyces, more preferably kluyveromyces marxianus, and/or Kluyveromyces lactis) as host cells.

When the polynucleotides of the present invention are expressed in higher eukaryotic cells, transcription will be enhanced if an enhancer sequence is inserted into the vector. Enhancers are cis-acting elements of DNA, usually about 10 to 300 base pairs in length, that act on a promoter to enhance transcription of a gene. Illustrative examples include the SV40 enhancer of 100 to 270 base pairs on the late side of the origin of replication, the polyoma enhancer on the late side of the origin of replication, and adenovirus enhancers, and the like.

It will be clear to those of ordinary skill in the art how to select appropriate vectors, promoters, enhancers and host cells.

Transformation of host cells with recombinant DNA can be performed using conventional techniques well known to those skilled in the art. When the host is a prokaryotic organism such as E. coli, competent cells capable of uptake of DNA can be harvested after exponential growth phase and treated with the CaCl₂ method, the procedures used are well known in the art. Another way is to use MgCl₂. If desired, transformation can also be performed by electroporation. When the host is an eukaryotic organism, the following DNA transfection methods can be used: calcium phosphate co-precipitation method, conventional mechanical methods such as microinjection, electroporation, liposome packaging, etc.

The obtained transformants can be cultured by conventional methods to express the polypeptides encoded by the genes of the present invention. The medium used in the culture can be selected from various conventional media depending on the host cells used. Cultivation is carried out under conditions suitable for growth of the host cells. After the host cells have grown to an appropriate cell density, the selected promoter is induced by an appropriate method (e.g., temperature switching or chemical induction) and the cells are cultured for an additional period of time.

The recombinant polypeptide in the above method can be expressed intracellularly, or on the cell membrane, or secreted outside the cell. If desired, recombinant proteins can be isolated and purified by various isolation methods utilizing their physical, chemical and other properties. These methods are well known to those skilled in the art. Examples of these methods include, but are not limited to: conventional renaturation treatment, treatment with protein precipitants (salting-out method), centrifugation, osmotic disruption, ultratreatment, ultracentrifugation, molecular sieve chromatography (gel filtration), adsorption chromatography, ion exchange chromatography, high performance liquid chromatography (HPLC) and various other liquid chromatography techniques and combinations of these methods.

Peptide Linker

The present invention provides a fusion protein, which may optionally contain a peptide linker. Peptide linker size and complexity may affect protein activity. In general, the peptide linker should be of sufficient length and flexibility to ensure that the two proteins connected have sufficient freedom in space to perform their function. At the same time, the influence of the formation of α helix or β sheet in the peptide linker on the stability of the fusion protein is avoided.

The length of the linker peptide is generally 0-20 amino acids, preferably 0-10 amino acids.

Pharmaceutical Composition

The present invention also provides a pharmaceutical composition. In a preferred example, the composition is a pharmaceutical composition, which contains the above-mentioned fusion protein, and a pharmaceutically acceptable carrier, diluent, stabilizer and/or thickener, and can be prepared into pharmaceutical forms such as lyophilized powders, tablets, capsules, syrups, solutions or suspensions.

“Pharmaceutically acceptable carrier or excipient” means: one or more compatible solid or liquid filler or gel substances, which are suitable for human use and which must be of sufficient purity and sufficient low toxicity. “Compatibility” as used herein means that the components of the composition can be blended with the active ingredients of the present invention and with each other without significantly reducing the efficacy of the active ingredients.

Compositions may be liquid or solid, such as powders, gels or pastes. Preferably, the composition is a liquid, preferably an injectable liquid. Suitable excipients will be known to those skilled in the art.

Some examples of pharmaceutically acceptable carriers are cellulose and its derivatives (such as sodium carboxymethyl cellulose, sodium ethyl cellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween®), wetting agents (such as Sodium lauryl Sulfates), colorants, flavors, stabilizers, antioxidants, preservatives, pyrogen-free water, etc.

The compositions may comprise physiologically acceptable sterile aqueous or anhydrous solutions, dispersions, suspensions or emulsions, and sterile powders for reconstitution into sterile injectable solutions or dispersions. Suitable aqueous and non-aqueous carriers, diluents, solvents or excipients include water, ethanol, polyols and suitable mixtures thereof.

Generally, these materials can be formulated in a non-toxic, inert and pharmaceutically acceptable aqueous carrier medium, usually at a pH of about 5-8, preferably at a pH of about 6-8, although pH can vary depending on the nature of the substance being formulated and the condition being treated. The formulated pharmaceutical compositions can be administered by conventional routes including, but not limited to, intraperitoneal, intravenous, or topical administration. The pharmaceutical composition is used for (i) preventing and/or treating infectious diseases; and/or (ii) preventing and/or treating autoimmune diseases; and/or (iii) preventing and/or treating osteoporosis or loss; and/or (iv) preventing and/or treating tumor-related diseases; and/or (v) inhibiting TNFa-induced apoptosis; and/or (vi) inhibiting TRAIL-induced apoptosis; and/or (vii) inhibiting RANKL-induced monocyte-macrophage differentiation; and/or (viii) inhibiting TNFa-induced immune activation; and/or (ix) inhibiting RANKL-induced osteoclastogenesis.

The Main Advantages of the Present Invention Include:

(a) The fusion protein of the present invention has a long half-life.

(b) The fusion protein of the present invention can not only improve inflammation in RA patients, but also prevent focal bone erosion and systemic bone loss caused by inflammation.

(c) The present invention finds for the first time that the fusion protein of the present invention can significantly (i) prevent and/or treat infectious diseases; and/or (ii) prevent and/or treat autoimmune diseases; and/or (iii) inhibit TNFa-induced apoptosis; and/or (iv) inhibit TRAIL-induced apoptosis; and/or (v) inhibit RANKL-induced monocyte-macrophage differentiation; and/or (vi) inhibit autoimmune disease induced osteoporosis and loss; and/or (vii) inhibit bone metastases.

The present invention will be further described below in conjunction with specific embodiments. It should be understood that these examples are only used to illustrate the present invention and not to limit the scope of the present invention. The experimental method of unreceipted specific conditions in the following examples, usually according to conventional conditions, such as people such as Sambrook, molecular cloning: conditions described in laboratory manual (New York: Cold Spring Harbor Laboratory Press, 1989), or according to manufacture conditions recommended by the manufacturer. Percentages and parts are weight percentages and parts unless otherwise specified. Unless otherwise specified, the reagents and materials in the examples of the present invention are all commercially available products.

### EXAMPLE 1 CONSTRUCTION OF IGG FC FUSION PROTEIN EXPRESSION PLASMID OF TNFR2 AND OPG

1. Construction of TNFR2-OPG-Fc fusion protein (hereinafter referred to as OTFc) expression plasmid

The human OPG gene sequence was derived from the human OPG full-length cDNA (Open Biosystems, MHS1011-7509022), and the human TNFR2 and human IgG Fc sequences were derived from the human TNFR2 (1-257)-Fc fusion protein gene and had been constructed.

In order to construct the OPG-TNFR2-Fc fusion protein expression vector, first, the polymerase chain reaction (PCR) method was used to synthesize the gene encoding OPG amino acids 1-194 using the human OPG full-length cDNA as the template (the primers at the 5′ and 3′ ends were primer-1 and primer-2, respectively. The primer sequences are shown in Table 1, the same below) and used the human TNFR2(1-257)-Fc fusion protein gene as a template to synthesize the gene encoding human TNFR2-Fc fusion protein (no protein signal peptide coding sequence 1-22, 5′ and 3′, the 5′ and 3′ primers are primer-3 and primer-4, respectively) and then the above two PCR fragments were connected by overlap extension PCR method to synthesize the complete gene encoding OPG(1-194)-TNFR2(23-257)-Fc fusion protein (5′ and 3′ primers are primer-1 and primer-4, respectively). The first 23 nucleotide sequences of primer-2 are complementary to the nucleotide sequence of primer-3, so that the two PCR fragments can be ligated during the overlap extension PCR process in the second step. Primer-1 contains restriction endonuclease Not I sequence, and primer-4 contains restriction endonuclease Xba I sequence for insertion into protein expression vector. The synthesized PCR fragments were separated by agarose gel electrophoresis and purified with DNA gel purification kit (QIAGEN). The OPG-TNFR2-Fc fusion protein gene synthesized by overlap extension PCR was first cloned into pCR2.1 vector (Invitrogen) with T/A vector cloning kit, and then the fusion protein gene was then cut from this vector with Not I and Xba I restriction enzymes and inserted into a mammalian cell expression plasmid also digested with Not I and Xba I enzymes, such as pcDNA3.1 (Invitrogen). The OPG (1-194)-TNFR2(23-257)-Fc DNA sequence inserted in pCR2.1 was confirmed by DNA sequencing.

2. Construction of TNFR2 (1-257)-OPG(22-194)-TNFR2(202-257)-Fc (hereinafter referred to as TOFc) fusion protein expression plasmid

In order to construct a TNFR2 (1-257)-OPG(22-194)-TNFR2(202-257)-Fc (hereinafter referred to as TNFR2-OPG-Fc) fusion protein expression vector, the polymerase chain reaction (PCR) method was used to synthesize the gene encoding amino acids 1-257 of TNFR2 (the 5′ and 3′ end primers are primer-5 and primer-6, respectively), and the gene encoding amino acids 22-194 of OPG (without the gene encoding the protein signal peptide 1-21, the primers at the 5′ and 3′ ends are primer-7 and primer-8, respectively) and the Fc-encoding gene (containing TNFR2 amino acids 202-257, the 5′ and 3′ primers are primer- 9 and primer-4, respectively), and then used the overlap extension PCR method to connect the above first and second PCR fragments respectively (the primers at the 5′ and 3′ ends are primer-5 and primer-8, respectively), and the second and third PCR fragments were ligated (the 5′ and 3′ end primers are primer-7 and primer-4, respectively), then, the two PCR fragments were connected by overlapping extension PCR method (the primers at the 5′ and 3′ ends were primer-5 and primer-4, respectively) to synthesize the full-length gene encoding the TNFR2(1-257)-OPG(22-194)-TNFR2(202-257)-Fc fusion protein OPG-TNFR2-Fc fusion protein. The synthesized fusion protein gene was first cloned into pCR-Blunt II-TOPO vector (Invitrogen) with T/A vector cloning kit, and then the fusion protein gene was excised from this vector with Not I and Xba I restriction enzymes and inserted into a mammalian expression plasmid that was also digested with Not I and Xba I, such as pcDNA3.1 (Invitrogen). The TNFR2 (1-257)-OPG(22-194)-TNFR2(202-257)-Fc DNA sequence inserted in the pCR-Blunt II-TOPO vector was confirmed by DNA sequencing.

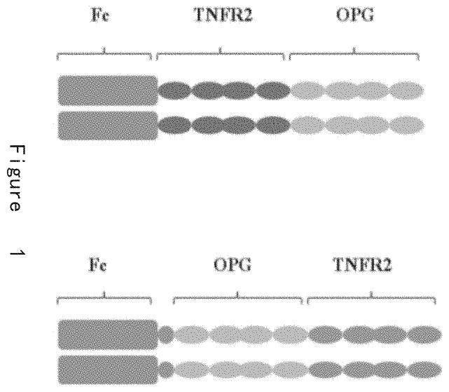

The schematic structural diagrams of the two fusion proteins constructed above are shown in FIG. 1.

TABLE 1

| Primer-1 | 5′-AGGTATAGCGGCCGCCACCATGAACA | SEQ |
|---|---|---|
| | ACTTGCTGTGCTGC-3′ | ID |
| | | NO.: |
| | | 6 |
| Primer-2 | 5′-GTAAATGCCACCTGGGCGGGCAATTT | SEQ |
| | TTGAGTTGATTCACTGTTTC-3′ | ID |
| | | NO.: |
| | | 7 |
| Primer-3 | 5′-TTGCCCGCCCAGGTGGCATTTAC-3′ | SEQ |
| | | ID |
| | | NO.: |
| | | 8 |
| Primer-4 | 5′-TGGTGGTGTCTAGAGACTCATTTACC | SEQ |
| | CGGAGACAGGGAGAGGC-3′ | ID |
| | | NO.: |
| | | 9 |
| Primer-5 | 5′-AAGCTTGCGGCCGCGAGCTCGGATCC | SEQ |
| | ACT-3′ | ID |
| | | NO.: |
| | | 10 |
| Primer-6 | 5′-GTCGCCAGTGCTCCCTTCAG-3′ | SEQ |
| | | ID |
| | | NO.: |
| | | 11 |
| Primer-7 | 5′-CTGAAGGGAGCACTGGCGACGAAACG | SEQ |
| | TTTCCTCCAAAGTACC-3′ | ID |
| | | NO.: |
| | | 12 |
| Primer-8 | 5′-TTTTTGAGTTGATTCACTGTTTC-3′ | SEQ |
| | | ID |
| | | NO.: |
| | | 13 |
| Primer-9 | 5′-GAAACAGTGAATCAACTCAAAAATCC | SEQ |
| | ACGTCCCCCACCCGG-3′ | ID |
| | | NO.: |
| | | 14 |

### EXAMPLE 2 CONSTRUCTION OF FUSION PROTEIN STABLE EXPRESSION CELL LINE

The host cell used to construct stable expression fusion protein cells is Chinese hamster ovary cell CHO-KS. CHO-KS is a CHO-K1 cell grown in a medium containing fetal bovine serum (FBS), after gradually reducing the FBS content in the medium until it is cultured in a FBS-free medium, and cells were finally acclimated to suspension grown in FBS-free OptiCHO medium (Invitrogen). The anti-neomycin gene in the pcDNA3.1 vector containing the fusion protein gene was replaced by the rat glutamine synthetase gene, and the fusion protein expression vector was transfected into CHO-KS cells by electrotransfection (Bio-Rad, Gene Pulser Xcell), after culturing the transfected cells for 24 hours, the transfected cells were screened and cultured on a 96-well culture plate by limiting dilution method. The selection medium was OptiCHO, 5 μg/ml recombinant human insulin and 10 μM methionine sulphoximine (MSX). Cells were cultured in a 37° C., 8% CO₂ incubator. After 3 weeks, cell culture medium for each well with a population of cells was analyzed by ELISA (alkaline phosphatase-conjugated goat anti-human IgG Fc antibody, Jackson ImmunoResearch Lab) and the cell populations with positive fusion protein expression were further expanded, detected by ELISA, and then expanded, and finally a stable cell populations with fusion protein expression is obtained.

### EXAMPLE 3 EXPRESSION AND PURIFICATION OF FUSION PROTEIN

The fusion protein cell line constructed in Example 2 was cultured and expanded to 2 liters. The culture supernatant was harvested, and the fusion protein was purified with Protein-A affinity chromatography column (POROS MabCapture A, Life Tech).

The purified fusion proteins were analyzed by reduced and non-reduced SDS-PAGE electrophoresis and HPLC-SEC (High Pressure Liquid-Molecular Sieve) (TSKgel G3000SWXL, TOSOH Bioscience).

Results and Analysis

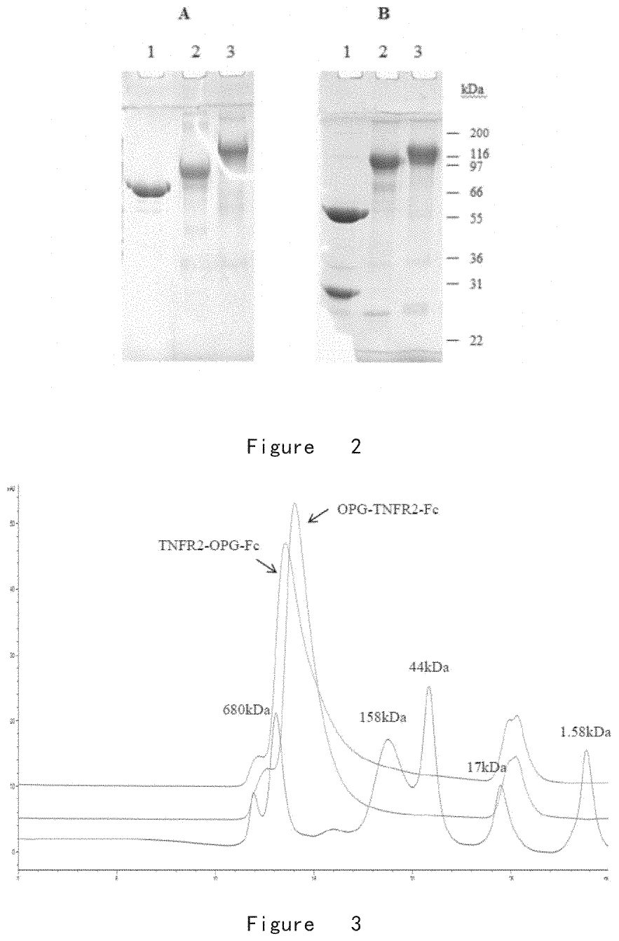

The OPG-TNFR2-Fc fusion protein is a homodimer with a theoretical molecular weight of about 142 kDa. The TNFR2-OPG-Fc fusion protein is a homodimer with a theoretical molecular weight of about 153 kDa. Non-reduced SDS-PAGE electrophoresis gel (FIG. 2A) shows that the molecular weights of these two homodimeric fusion proteins are larger than that of human immunoglobulin IgG1, and are close to their respective theoretical values. FIG. 2B shows the result of reduced SDS-PAGE electrophoresis gel of fusion protein single chain.

FIG. 3 shows the analysis results of HPLC-SEC (molecular sieve chromatography) of OPG-TNFR2-Fc fusion protein and TNFR2-OPG-Fc fusion protein. This result shows that the fusion protein peak is located around 600 kDa. OPG and TNFR2 each has 4 cysteine-rich regions (CRDs), and 8 CRDs are linked together to make the fusion protein molecule form a rod-like structure, which shows a molecular weight larger than that of the standard protein with a compact structure in molecular sieve chromatography column analysis.

### EXAMPLE 4 IN VITRO BINDING STUDY WITH TNFA

The in vitro specific binding activity of the fusion protein to TNFa was studied by ELISA method. Recombinant human TNFa (rhTNFa, SinoBiological) was dissolved in PBS (pH 7.0) solution, added to a 96-well ELISA plate, and kept in a refrigerator at 4° C. overnight. The next day, free rhTNFa was washed away with PBST (PBS containing 0.05% Tween-20), and added 3% BSA in PBST blocking solution. Serial dilutions of different concentrations of fusion protein were added to the blocked ELISA plate, the fusion protein bound to TNFa was detected with alkaline phosphatase-conjugated goat anti-human IgG Fc antibody (Jackson ImmunoResearch Lab), and the substrate (PNPP) was added for color development. Read the plate with a microplate reader at a wavelength of 405 nm/655 nm.

Results and Analysis

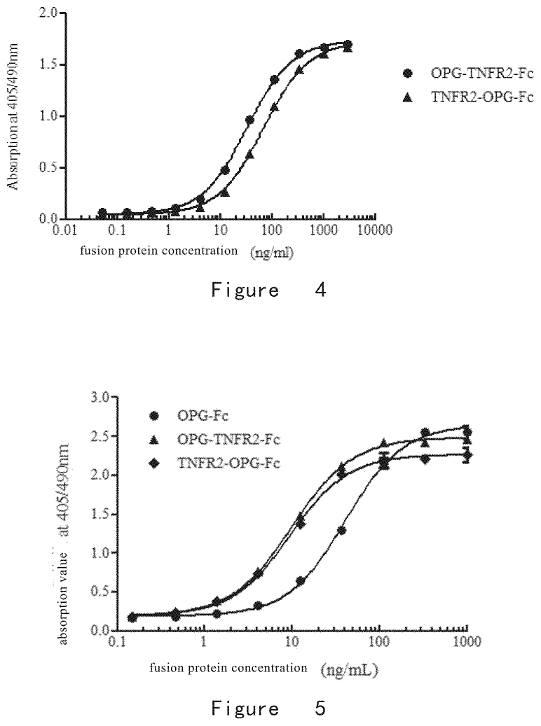

The results of in vitro binding ELISA studies of OPG-TNFR2-Fc fusion protein and TNFR2-OPG-Fc fusion protein and rhTNFa are provided, as shown in FIG. 4. The results show that the two fusion proteins can specifically bind to rhTNFa with EC₅₀ of 33 ng/ml and 68 ng/ml, respectively.

### EXAMPLE 5 IN VITRO BINDING TO RANKL

The in vitro specific binding activity of fusion protein to RANKL was studied by ELISA method. Dissolve recombinant human RANKL-murine Fc fusion protein (Sino Biological Inc.) in PBS (pH 7.0) to 1 μg/mL, add 50 μL to each well of a 96-well ELISA plate, and in the refrigerator at 4° C. overnight. The next day, washed 3 times with PBST (PBS containing 0.05% Tween-20), adding 200 μL to each well, and adding PBST blocking solution containing 3% BSA. The blocking solution was removed and serial dilutions of the following 3 fusion proteins, OPG-TNFR2-Fc, TNFR2-OPG-Fc and OPG-Fc (Sino Biological Inc., as control samples) were added to the ELISA plate. The fusion protein bound to RANKL was detected with an alkaline phosphatase-conjugated goat anti-human IgG Fc antibody (Jackson ImmunoResearch Lab), and a substrate (PNPP) was added for color development, and read the plate with a microplate reader at dual wavelengths 405 nm/490 nm.

Results and Analysis

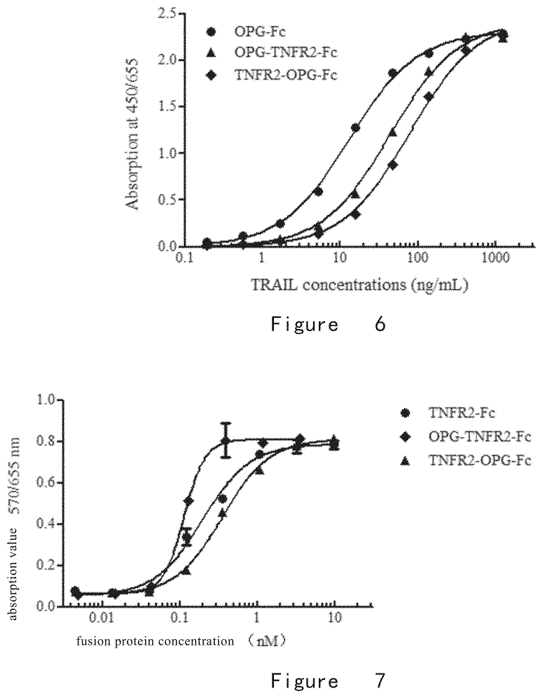

The experimental results show (FIG. 5) that all three fusion proteins can specifically bind to human RANKL, but OPG-TNFR2-Fc and TNFR2-OPG-Fc have stronger binding activities to RANKL than OPG-Fc, and their binding activity EC₅₀ are 9.9 ng/mL, 9.4 ng/mL and 40 ng/mL, respectively. There is no significant difference in the binding activity of OPG-TNFR2-Fc and TNFR2-OPG-Fc to RANKL.

### EXAMPLE 6 IN VITRO BINDING TO TRAIL

The in vitro specific binding activity of fusion protein to TRAIL was studied by ELISA method. The OPG-TNFR2-Fc, TNFR2-OPG-Fc and OPG-Fc (Sino Biological Inc.) fusion proteins were diluted with PBS to 1 μg/mL, respectively, and 50 μL was added to each well of a 96-well ELISA plate, and in the refrigerator at 4° C. overnight. The next day, washed 3 times with PBST (PBS containing 0.05% Tween-20), and added 200 □μL of 3% BSA-containing PBST blocking solution to each well. The blocking solution was removed, and serially diluted rhTRAIL (Histidine tag, R&D Systems) was added to the blocked ELISA plate. The rhTRAIL bound to the fusion protein was detected with horseradish peroxidase (HRP)-conjugated mouse anti-Histidine antibody (Sino Biological Inc.), adding substrate (TMB) for color development, and reading the plate with a microplate reader at dual wavelengths of 405 nm/490 nm.

Results and Analysis

The experimental results show (FIG. 6) that rhTRAIL can specifically bind to the three fusion proteins. Although the binding activities of OPG-TNFR2-Fc and TNFR2-OPG-Fc to TRAIL are weaker than that of OPG-Fc, the EC₅₀ of their binding activities is respectively 43 ng/mL, 78 ng/mL and 14 ng/mL. OPG-TNFR2-Fc binds TRAIL slightly more than TNFR2-OPG-Fc.

### EXAMPLE 7 IN VITRO BIOLOGICAL ACTIVITY STUDY OF TNFR2

The TNFR2 biological activity of the fusion protein was assayed by an in vitro neutralization TNFa biological activity assay. TNFa biological activity was detected by mouse fibroblast L929 cytotoxicity. rhTNFa was mixed with serial dilutions of different concentrations of fusion proteins, OPG-TNFR2-Fc, TNFR2-OPG-Fc and TNFR2 (Etanercept, Biogen Inc, as a control sample) into L929 cells and the viability of L929 cells was detected by Crystal Violet Staining method after 20 hours of culture in a cell incubator.

Results and Analysis

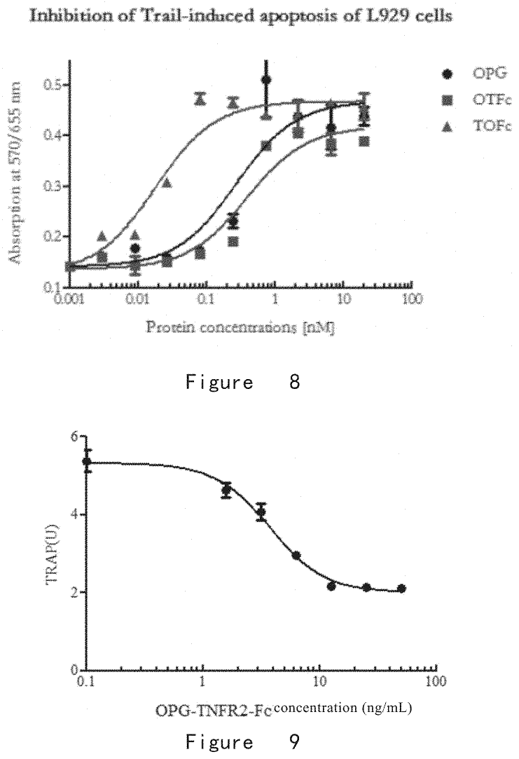

Both OPG-TNFR2-Fc fusion protein and TNFR2-OPG-Fc fusion protein can inhibit rhTNFa-induced apoptosis of L929 cells (FIG. 7). OPG-TNFR2-Fc inhibits TNFa-induced apoptosis of L929 cells (EC₅₀=0.11 nM) more than Etanercept (EC₅₀=0.19 nM) and also stronger than TNFR2-OPG-Fc (EC₅₀=0.35 nM).

### EXAMPLE 8 IN VITRO BIOLOGICAL ACTIVITY STUDY OF OPG

1. Inhibition of TRAIL-Induced Apoptosis in L929 Cells

Recombinant human TRAIL (R&D Systems Inc.) was mixed with serial dilutions of fusion protein at different concentrations and added to L929 cells. After culturing in a cell incubator for 20 hours, the viability of L929 cells was detected by Crystal Violet Staining method. Recombinant human OPG-Fc fusion protein (Novo Protein)

Results and Analysis

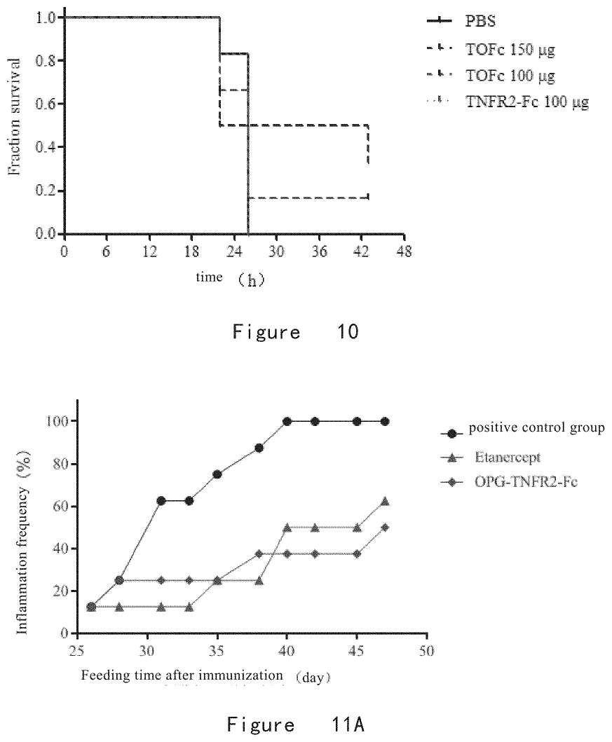

The ability of TNFR2-OPG-Fc fusion protein to inhibit rhTRAIL-induced apoptosis of L929 cells is significantly stronger than that of OPG-Fc and OPG-TNFR2-Fc (FIG. 8). The IC50 of TNFR2-OPG-Fc fusion protein in inhibiting rhTRAIL-induced apoptosis of L929 cells is 0.02 nM, and the inhibitory activities of OPG-Fc and OPG-TNFR2-Fc are comparable, with IC50s of 0.27 nM and 0.39 nM, respectively.

2. Inhibition of RANKL-Induced Differentiation of Mouse Monocyte-Macrophage RAW264.7

The activity of RANKL to induce the differentiation of mouse monocyte-macrophage RAW264.7 and the activity of OPG-TNFR2-Fc to inhibit the differentiation of RANKL-induced cells were detected by tartrate-resistant acid phosphate staining method (TRAP). Raw264.7 cells (Cell Bank of the Type Culture Collection, Chinese Academy of Sciences) were cultured in DMEM+10% FBS medium. One day before the experiment, adding 5×10³ cells of 100 μL per well of a 96-well cell culture plate. After culturing cells for 24 hours, the supernatant was emptied, and adding 200 μL □-MEM medium+10% FBS containing 20 ng/mL RANKL and different concentrations of OPG-TNFR2-Fc fusion protein to each well. After culturing the cells for 2 days, the supernatant was emptied, and adding freshly prepared same concentration of RANKL and corresponding concentration of OPG-TNFR2-Fc, and continued to culture cells for 3 days, the supernatant was emptied, wash the cells twice with PBS solution, then add 100 μL of 1% triton X-100 solution (diluted in PBS) to each well to lyse the cells, take the supernatant after high-speed centrifugation, and use TRAP detection kit (Biyuntian Biotechnology Co., Ltd.) to detect the content of TRAP in cell lysates.

Results and Analysis

The experimental results show (FIG. 9) that with the increase of the concentration of OPG-TNFR2-Fc fusion protein, the content of acid phosphatase produced by RANKL-induced RAW264.7 cells is decreased, indicating that OPG-TNFR2-Fc fusion protein can inhibit RANKL-induced differentiation of mouse monocyte-macrophage RAW264.7.

### EXAMPLE 9 MOUSE MODEL STUDY

1. Inhibition of LPS-Induced Septic Shock Death in Mice

Detecting the effect of fusion protein on LPS-induced septic shock death in mice to study the in vivo biological activity of fusion protein TNFR2. Twenty-four Balb/c mice aged 8-10 weeks were divided into 4 groups, 6 mice for each group (half male and half male). Each mouse was intraperitoneally injected with 1 mg LPS, followed by intravenous injection of different doses of TNFR2-OPG-Fc fusion protein and Etanercept (Pfize). The state of the mice was observed in the following 2 days, and the time of death of the mice was recorded.

Results and Analysis

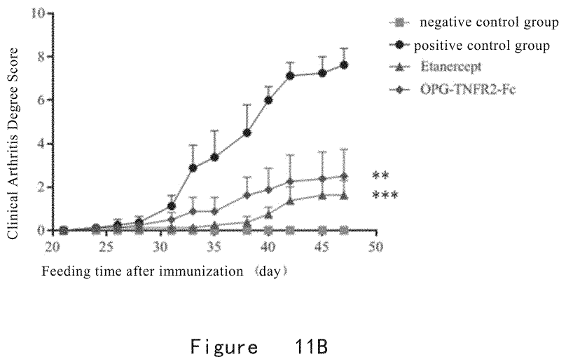

After the lethal dose of 1 mg LPS/mouse was injected, all the mice in the control group died within 26 hours. In 2 groups of mice injected with TNFR2-OPG-Fc fusion protein 100 μg/mice and 150 μg/mice, 2 and 2 mice were survived respectively, and 1 and 2 mice were survived at 46 hours. The mice injected with TNFR2-Fc (Etanercept) 100 μg/mice (the dose equivalent to TNFR2-OPG-Fc 150 μg/mice) also died within 26 hours (FIG. 10).

2. CIA-Induced Mouse Arthritis Model

The methods for establishing the CIA mouse arthritis model were respectively referred to Feige et al., Cell Mol Life Sci, 57:1457-1470, 2000 and Schett et al., Arthritis Rheum. 52:1604-1611, 2005. CIA model mice were randomly divided into 4 groups, namely negative control group (group 1, no immunization, no drug administration), positive control group (group 2, immunization but no drug administration), and Etanercept group (group 3, 25 mg/kg) and the OGP-TNFR2-Fc fusion protein group (group 4, 30 mg/kg, molar content equivalent to 25 mg/kg of Etanercept) with 8 mice per group. Mice in groups 1-3 were immunized twice on D1 and D22, respectively, and then mice in groups 3 and 4 were intraperitoneally injected with the corresponding fusion proteins, 3 times a week, for a total of 11 times.

The clinical symptoms of mice were detected as follows:

At the end of the second immunization of D22 animals, clinical joint scoring was started, and clinical scoring was performed three times a week. The clinical scoring criteria are as follows:

| Paw | |
|---|---|
| Score | Clinical Observation |
| 0 | normal. |
| 1 | Mild but marked redness and swelling of the ankle or wrist joints, |
| | or marked redness and swelling limited to individual toes, |
| | regardless of the number of toes |
| 2 | Moderate redness and swelling of ankle or wrist joints, redness and |
| | swelling of 2 different knuckle parts |
| 3 | Moderate redness and swelling of the entire paw (including the |
| | toes) |
| 4 | Severe inflamed and redness and swelling limbs, involving |
| | multiple joints |

The degree of infiltration of inflammatory cells in the ankles and vertebra of mice in each group was evaluated. Serum was collected on D35 and 24 hours after the last dose, respectively. The right hind paw ankle joint was collected at the end point, fixed with 4% paraformaldehyde, sections were stained with HE after decalcification for histopathological scoring. The parameters of cellular infiltration, bone erosion and cartilage damage were graded separately.

Bone mineral density (BMD) evaluation. At the end of the experiment, the left posterior bone and vertebra were dehydrated by soaking in 70% ethanol, and the bone density was detected by micro-CT.

Results and Analysis

The results of this experiment show that at the end of the experiment, all the mice in the positive control group show symptoms of arthritis in their paws, while the incidence of inflammation in mice in the OPG-TNFR2-Fc and etanercept groups is between 50-60% (FIG. 11A). The results of clinical scoring of mouse paw inflammation show the average degree of inflammation in the paws of the mice in the OPG-TNFR2-Fc group is significantly lower than that of the mice in the positive control group, and is comparable to the degree of inflammation exhibited by the mice in the etanercept group (FIG. 11B). The results of this experiment prove that OPG-TNFR2-Fc fusion protein has better function to protect and reduce the degree of CIA-induced arthritis in mice.

### EXAMPLE 10 STUDY ON THE EFFECT OF OPG-TNFR2-FC FUSION PROTEIN ON BONE ABSORPTION IN MACACA FASCICULARIS

The effect of fusion protein on bone absorption in macaca fascicularis was investigated by studying the effect of OPG-TNFR2-Fc fusion protein on the concentration of tartrate-resistant acid phosphatase-5b (TRAP-5b) in macaca fascicularis serum. TRAP-5b is a metabolite in the process of bone absorption and is used as a biomarker for bone absorption. Six male macaca fascicularis (3-5 years old) were divided into 3 groups. One group was given 15 mg/kg OPG-TNFR2-Fc fusion protein intravenously, and the other two groups were injected subcutaneously with 5 mg/kg and 15 mg/kg OPG-TNFR2-Fc fusion protein, respectively. Blood was taken to retain serum at the following times, pre-dose, 24 hours, 48 hours and 168 hours post-dose. The concentration of TRAP-5b in serum was detected with TRAP-5b detection kit (Shanghai Lanji Biotechnology Co., Ltd.).

Results and Analysis

Table 2 shows studies that reduce the concentration of the bone absorption marker TRAP-5b in macaca fascicularis serum.

TABLE 2
TRAP-5b

| | | | Before [symbol] | concentration (ng/mL) | | |
|---|---|---|---|---|---|---|
| | Mode of Administration | | admini- | (changes from pre-dose) [symbol] | | |
| Group | and Dosage [symbol] | | stration [symbol] | 24 h | 48 h | 168 h |
| 1 | subcutaneous | macaca | 54 | 44 (−19%) | 49 (−9%) | 52 (−4%) |
| | admini- | fascicularis- | 53 | 47 (−11%) | 42 (−21%) | 52 (−2%) |
| | stration [symbol] | 1 [symbol] | | | | |
| | mg/kg | macaca | | | | |
| | | fascicularis- | | | | |
| | | 2 [symbol] | | | | |
| 2 | subcutaneous | macaca | 40 | 28 (−30%) | 36 (−10%) | 38 (−5%) |
| | admini- | fascicularis- | 31 | 24 (−23%) | 32 (3%) | 35 (13%) |
| | stration [symbol] | 1 [symbol] | | | | |
| | mg/kg | macaca | | | | |
| | | fascicularis- | | | | |
| | | 2 [symbol] | | | | |
| 3 | intravenous | macaca | 54 | 44 (−19%) | 51 (−6%) | 49 (−9%) |
| | admini- | fascicularis- | 62 | 37 (−40%) | 52 (−16%) | 45 (−27%) |
| | stration [symbol] | 1 [symbol] | | | | |
| | mg/kg | macaca | | | | |
| | | fascicularis- | | | | |
| | | 2 [symbol] | | | | |

| | | | | |
|---|---|---|---|---|
| mean [symbol] | 49 | 37 (−24%) | 44 (−11%) | 45 (−8%) [symbol] |

[symbol]

indicates data missing or illegible when filed

The results of the study (Table 2) show that 24 hours after administration of the OPG-TNFR2-Fc fusion protein, the concentration of TRAP-5b in the serum of all macaca fascicularis is reduced, ranging from 11% to 40%. In the subcutaneous administration group, the degree of decrease in TRAP-5b concentration is proportional to the dose administered. Mean serum TRAP-5b concentrations in all macaca fascicularis is decreased by 24%, 11%, and 8% after 24, 48, and 168 hours post-dose, respectively. This study shows that OPG-TNFR2-Fc fusion protein can inhibit bone absorption in macaca fascicularis.

All publications mentioned herein are incorporated by reference as if each individual document was cited as a reference, as in the present application. It should also be understood that, after reading the above teachings of the present invention, those skilled in the art can make various changes or modifications, equivalents of which falls in the scope of claims as defined in the appended claims.

1. A fusion protein comprising the following elements fused together: (a) a TNF receptor or an active fragment thereof; (b) OPG or an active fragment thereof; and optionally (c) Fc fragment.

2. The fusion protein of claim 1, wherein the TNF receptor is selected from the group consisting of TNFR2, TNFR1, and a combination thereof.

3. An isolated polynucleotide encoding the fusion protein of claim 1.

4. A vector, which contains the polynucleotide of claim 3.

5. A host cell, wherein the host cell contains the vector of claim 4, or the polynucleotide of claim 3 is integrated into its genome.

6. A method for producing the fusion protein of claim 1, the method comprising the steps of:
Under conditions suitable for expression, culturing the host cell of claim 5, thereby expressing the fusion protein; and/or
Isolating or purifying the fusion protein.

7. A pharmaceutical composition comprising the fusion protein of claim 1 and a pharmaceutically acceptable carrier thereof.

8. Use of the fusion protein of claim 1, the polynucleotide of claim 3, the vector of claim 4, and the host cell of claim 5 for the preparation of a composition or preparation for (i) preventing and/or treating infectious diseases; and/or (ii) preventing and/or treating autoimmune diseases; and/or (iii) preventing and/or treating osteoporosis or loss; and/or (iv) preventing and/or treating tumor-related diseases.

9. A method for inhibiting cell apoptosis, comprising the steps of:
In the presence of the fusion protein of claim 1, culturing a cell, thereby inhibiting cell apoptosis.

10. A method for inhibiting the differentiation of mononuclear macrophages, comprising the steps of:
In the presence of the fusion protein of claim 1, culturing a mononuclear macrophage, thereby inhibiting the differentiation of the mononuclear macrophage.
